(19) United States
(12) Patent Application Publication
(10) Pub. No.: US 2007/0163601 A1
(43) Pub. Date: Jul. 19, 2007

(54) Apparatus and method for reversible male and female contraceptive implants

(76) Inventors: Neil Pollock, Vancouver (CA); David Fishcell, Fair Haven, NJ (US); Robert Fischell, Dayton, MD (US); John Burton, Minnetonka, MN (US)

Correspondence Address: BECK AND TYSVER P.L.L.C., 2900 THOMAS AVENUE SOUTH, SUITE 100, MINNEAPOLIS, MN, 55416, US

(21) Appl. No.: 11/333,695

(22) Filed: Jan. 17, 2006

(51) Int. Cl. A61F 6/02

(52) U.S. Cl. 128/843

(57) ABSTRACT

The device includes tethered plugs that may be reversibly inserted into the VAS to provide contraception. The method includes a procedure for inserting and removing the device.

### FIELD OF THE INVENTION

This invention is in the field of implantable devices to create reversible blockages of ducts within a human, and more specifically to the field of male and female contraceptive devices.

### BACKGROUND OF THE INVENTION

In U.S. Pat. No. 6,513,528 Burton et al. describe an implantable Intra-Vas Device (IVD) for reversible male sterilization. The device is a silicone rubber sock which when stretched over an insertion wire has a diameter small enough to allow it to be inserted into the vas deferens (hereinafter, the “vas”). Two of these prior art IVDs are typically used because study has shown that two obstructions with a “dead space” in between works best in preventing the flow of sperm through a vas. Because the range of expansion of silicone rubber is limited, the prior art IVD must be selected to be slightly larger than the inside diameter of the vas, requiring at least three different sizes to fit the general male population.

There are also some data suggesting that a “closed procedure” with simple blockage of the vas leads to more complications than an “open” procedure in which the vas wall is penetrated, the section of the vas toward the urethra (hereinafter, “u-section”) is blocked and section of the vas toward the testes (hereinafter, “t-section”) is left open so that sperm are free to flow the penetration into the scrotum, but not out through the urethra.

### SUMMARY OF THE INVENTION

The present invention is an improvement of the IVD that is adapted for both male and female reversible sterilization. The present invention provides dual or multiple duct blockages, separated by dead space(s), within a single inserted device. The present invention IVD is a “one size fits all” device using an expandable material having a much greater range of expansion than silicone rubber. A preferred embodiment uses a hydrophilic polymer foam such as polyvinyl alcohol (PVA) as the expandable material. PVA is also a good choice because it is currently approved by the United States Food and Drug Administration (FDA) for use in humans. PVA when dried can be compressed to 10% of its normal volume. The present invention device would typically use two PVA plugs connected together with a thin rod oriented longitudinally within the vas thereby creating a space between the plugs. The present invention device can be delivered by pushing it out from inside an introducer tube that is inserted into the vas, or it can be delivered over an insertion wire as in the prior art device.

Other hydrophilic materials that can expand in vivo and are useful in the practice of this invention include polyvinylpyrrolidone, polyethylene glycol, karaya gum, carboxy methyl cellulose, hyaluronic acid, dextran, polyacrylic acid, and other organic polymers containing carboxylic acid groups or their salts. Cross-linked hydrophilic polymers or hydrogels are particularly desirable. These should be insoluble yet still capable of expanding up to 1000% (like PVA) due to the absorption of water. Cross-linking may be by chemical means or by physical means. The hydrogels disclosed in U.S. Pat. Nos. 3,867,329 and 4,480,642 can also be useful in this invention. Reference can also be made to G.B. 2,139,989A for suitable cross-linked polymeric compositions.

The present invention IVD would be available in both closed and open embodiments. In a male, the closed end IVD is completely inserted into the vas and block it in two places. Like the prior art IVD, the closed end IVD can be removed to restore fertility.

The open end IVD includes an axial lumen to allow sperm to flow out of the vas through a shunt into the scrotum while preventing any flow into the u-section of the vas. If the IVD were removed, the sperm would flow again from the testes to the urethra through the vas. An optional suture could be used to repair the hole in the vas wall left by an extension of the open end IVD through the vas wall into the scrotum.

The method of use for the present invention IVD is also novel. By use of a ring clamp to fix the vas and dissecting forceps to isolate and expose the vas, each of the two vasa can be pulled one at a time, out of the scrotum. The present invention IVD can then be directly inserted into the vas through a small hole.

The present invention device is also applicable to female reversible contraception.

A removable Intra-Fallopian Tube Device (IFD) having a similar design to the closed end IVD is pushed out of a delivery tube to block a fallopian tube. The delivery catheter for such an IFD is typically longer than the delivery tube for an IVD and would typically be combined or used with an endoscope allowing insertion through the uterus via vaginal/cervical access.

It is an object of the present invention to have a single Intra-Vas Device (IVD) that is designed to block the vas in two places with a “dead space” in between.

Another object of the present invention is to have an IVD that uses expandable polymer foam or other expandable material to form the blockages in the vas.

Another object of the present invention is to have an IVD that uses polyvinyl alcohol (PVA), an expandable foam, as the expandable material.

Still another object of the present invention is to have an open end IVD that shunts sperm into the scrotum thereby preventing it from flowing to the urethra.

Yet another object of the present invention is to have an IVD that is deliverable by pushing it out of an introducer tube.

Yet another object of the present invention is a method to introduce the IVD through a non-damaging minimally invasive pinpoint opening in the vas without the use of a scalpel or scissors and only the tip of the dissecting forceps or larger gauge needle used as an entry device to the vas.

Yet another object of the present invention is to have a female Intra-Fallopian Tube Device (IFD) that uses an expandable material, such as an expandable polymer foam, to block a fallopian tube for reversible female contraception.

These and other objects and advantages of this invention will become obvious to a person of ordinary skill in this art upon reading of the detailed description of this invention including the associated drawings.

### BRIEF DESCRIPTION OF THE DRAWINGS

Throughout the several drawings like numerals indicate identical structure wherein,

FIG. 1 is an illustration of the prior art Intra-Vas Device (IVD) delivery system.

FIG. 2 is a transverse cross section of the present invention closed end IVD in its pre-deployment shape.

FIG. 3 is a transverse cross section of the present invention closed end IVD in its expanded state.

FIG. 4 is a transverse cross section of a first embodiment of a delivery system for the present invention closed end IVD.

FIG. 5 is a transverse cross section showing the present invention closed end IVD inside the vas. FIG. 6 is a transverse cross section of the present invention open end IVD in its pre-deployment shape.

FIG. 7 is a transverse cross section of the present invention open end IVD in its expanded state.

FIG. 8 is a transverse cross section showing the configuration whereby the present invention open end IVD shunts sperm from the vas into the scrotum while blocking flow to the urethra

FIG. 9 is the transverse cross section of the delivery system for the present invention Intra-Fallopian Device (IFD).

FIG. 10 is the transverse cross section of the IFD implanted at the ostium of a fallopian tube.

FIG. 11 is the transverse cross section of an alternate embodiment of the IFD implanted at the ostium of a fallopian tube.

### DETAILED DESCRIPTION OF THE DRAWINGS

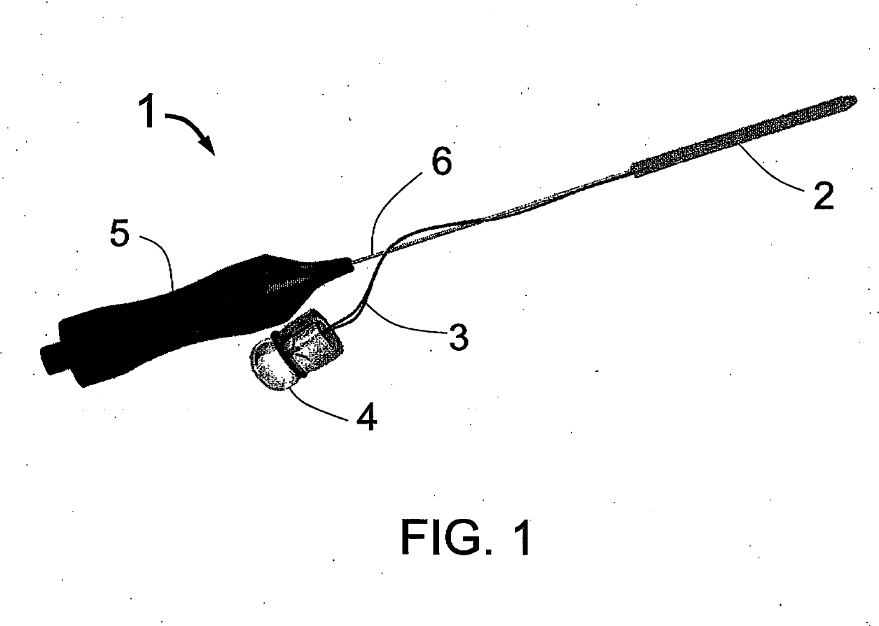

FIG. 1 is an illustration of the prior art Intra-Vas Device (IVD) delivery system 1 with IVD 2. Attached to the IVD is an attachment cord 3 with cord handle 4. The delivery system 1 also includes the introducer needle 6 with handle 5. To use the delivery system 1, the IVD 2 is stretched to a increased length with reduced diameter over the needle 6 by pulling back on the cord handle 4. With the IVD 2 stretched, the IVD 2 is inserted into the vas through a surgically created hole. When the cord handle 4 is released, the IVD 2 expands to fill the vas duct. The cord can then be sutured to the outside of the Vas to prevent migration of the IVD 2. For best effect, two IVDs 2 are typically implanted with a dead space in between into the vas.

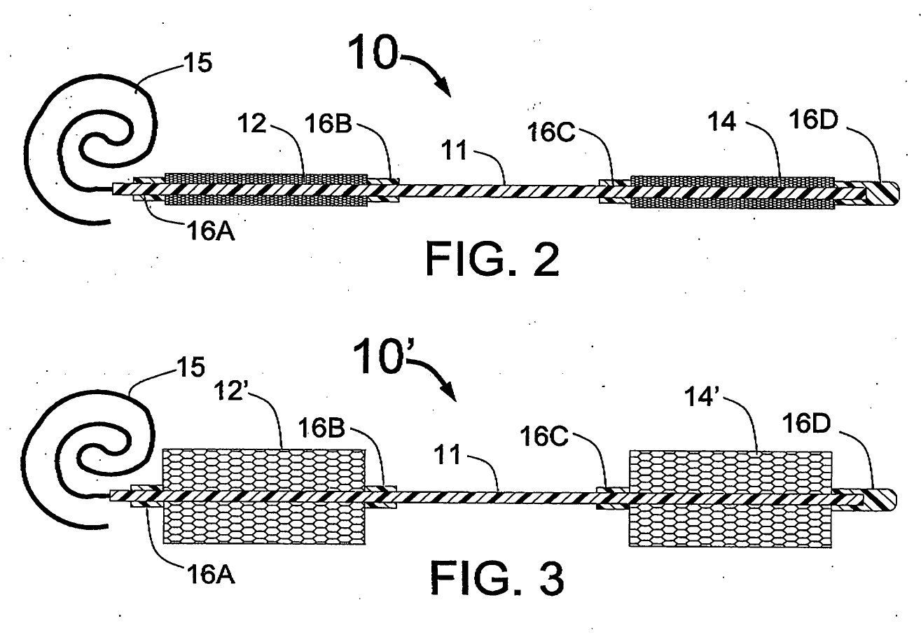

FIG. 2 is a transverse cross section of the present invention closed end IVD 10 in its pre-deployment shape. The IVD 10 is flexible molded plastic rod 11 with a cord 15 attached to the proximal end. Attached to the rod 11 are two central raised portions 16B and 16C. At its urethra end, the IVD 10 includes an expandable cylinder 12 inserted over the rod 11 and held in place between the stop 16A and the central raised portion 16B. At its testes end, IVD 10 includes an expandable cylinder 14 inserted over the rod 11 and held in place between the cap 16D and the central raised portion 16C.

The rod 11, the raised portions 16B and 16C, the cap 16A and the stop 16D are typically made from one or more biocompatible materials such as those used in angioplasty catheters. These include polymers such as urethane, nylon, polyimid and TEFLON. The cord 15 would typically me made from a biostable material such as those used in permanent sutures, e.g. nylon.

The expandable cylinders 12 and 14 may be any biocompatible expandable polymer; the preferred embodiment of the present invention would utilize a gradually expanding hydrophilic foam material. The gradually expanding hydrophilic material can be any biologically compatible material such as hydrogels which are capable of expanding slowly when water is absorbed therewithin. Among the hydrogels, which are employable in the context of this invention are those utilized heretofore in cervical dilators, or in cervical devices such as described in U.S. Pat. No. 3,867,329. Known slowly expanding dilators such as laminaria digitata or japonica can also be utilized.

Among the hydrophilic materials useful in the practice of this invention are polyvinylpyrrolidone, polyethylene glycol, karaya gum, carboxy methyl cellulose, hyaluronic acid, dextran, polyacrylic acid, polyvinyl alcohol and other organic polymers containing carboxylic acid groups or their salts. Cross-linked hydrophilic polymers or hydrogels are particularly desirable. These should be insoluble yet still capable of expanding up to 1000% due to the absorption of water. Cross-linking may be by chemical means or by physical means. The hydrogels disclosed in U.S. Pat. Nos. 3,867,329 and 4,480,642 can also be useful in this invention. Reference can also be made to G.B. 2,139,989A for suitable cross-linked polymeric compositions.

FIG. 3 is a transverse cross section of the present invention closed end IVD in its post-deployment shape 10′. After deployment into the vas, the foam cylinders 12 and 14 of FIG. 2 will expand to become the expanded foam cylinders 12′ and 14′ of FIG. 3. The cord 15 is typically sutured to the vas to prevent migration of the IVD 10 after deployment and to facilitate removal of the IVD 10 when and if sterilization is to be reversed.

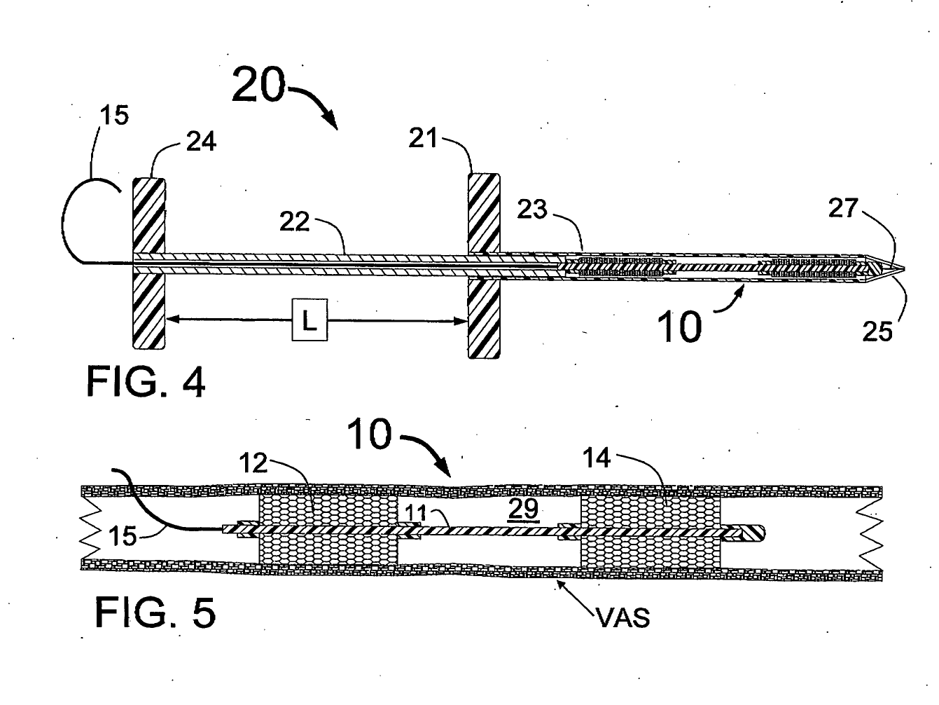

FIG. 4 is a transverse cross section of the pre-deployment configuration of the present invention IVD delivery system 20. During the process of insertion, the IVD has an insertion end to be positioned toward the testes, and a tail end to be positioned toward the urethra. The IVD 10 is housed within a delivery sheath 23 with handle 21 and slit tapered insertion end 25 with slit 27 shown. A pusher tube 22 with handle 24 provides the means of delivery of the IVD 10 into the vas. Specifically, once access to the vas is available, the tapered slit insertion end 25 of the sheath 23 is inserted into the vas. There are two ways in which the IVD 10 can then be delivered.

The first method requires that the sheath 23 be advanced into the vas until the handle 21 is just proximal to the opening into the vas. At this time, the handle 21 is pulled toward the handle 24 of the pusher tube 22. This will retract the sheath 23 leaving the IVD 10 inside the vas. The pusher tube 22 and sheath 23 can then be pulled over the cord 15, which is located at the tail (urethra) end of the IVD, and removed. The cord 15 can then be sutured to the vas and the procedure can be completed.

The second method requires that the sheath 23 be advanced into the vas until the slit tapered insertion end 25 is distal to the opening into the vas. At this time, the handle 24 is pushed toward the handle 21 of the sheath 23. This will push the IVD 10 out of the sheath 23 inside the vas. The pusher tube 22 and sheath 23 are then pulled over the cord 15 and removed. The cord 15 can then be sutured to the Vas and the procedure can be completed.

The two handles 21 and 24 are separated by the distance L which is typically greater than the length of the IVD 10 so that the length of the pusher tube 22 is sufficient to push the IVD 10 completely out of the sheath 23 into the vas. The handles 21 and 24 and the sheath 23 are typically made from one or more biocompatible materials such as those used in angioplasty catheters. These include polymers such as urethane, nylon, polyimid and TEFLON. The pusher tube 22 can be made from plastic or a metal such as aluminum or stainless steel.

FIG. 5 is a transverse cross section showing the closed end IVD 10 inside the vas. The expandable cylinders 12 and 14 mounted over the flexible rod 11 are designed to adapt to the individual vas, expanding to fill the particular diameter. Thus the cylinders 12 and 14 form blockages to sperm in two places with a dead space 29 in between. The cord 15 which extends through the vas wall typically at the side of insertion of the IVD 10 also serves the important function when and if the IVD 10 is to be removed to reverse sterilization. To remove the IVD 10 a hole in the vas is made at the location where the cord 15 goes through the vas wall and the cord 15 is pulled to remove the IVD 10 from the vas.

FIG. 6 is a transverse cross section of the present invention open end IVD in its pre-deployment shape. The IVD 30 is a flexible molded plastic tube 31 with lumen 38. Attached to the tube 31 are two central raised portions 36B and 36C. At the urethra end, the IVD 30 includes a expandable cylinder 12 inserted over the tube 31 and held in place between the stop 36A and the central raised portion 36B. At the testes end, the IVD 30 includes a expandable foam cylinder 34 inserted over the tube 31 and held in place between the cap 36D and the central raised portion 36C. The cord 35 is attached to the IVD 30 at a location outside of the expandable foam cylinder at the urethra end.

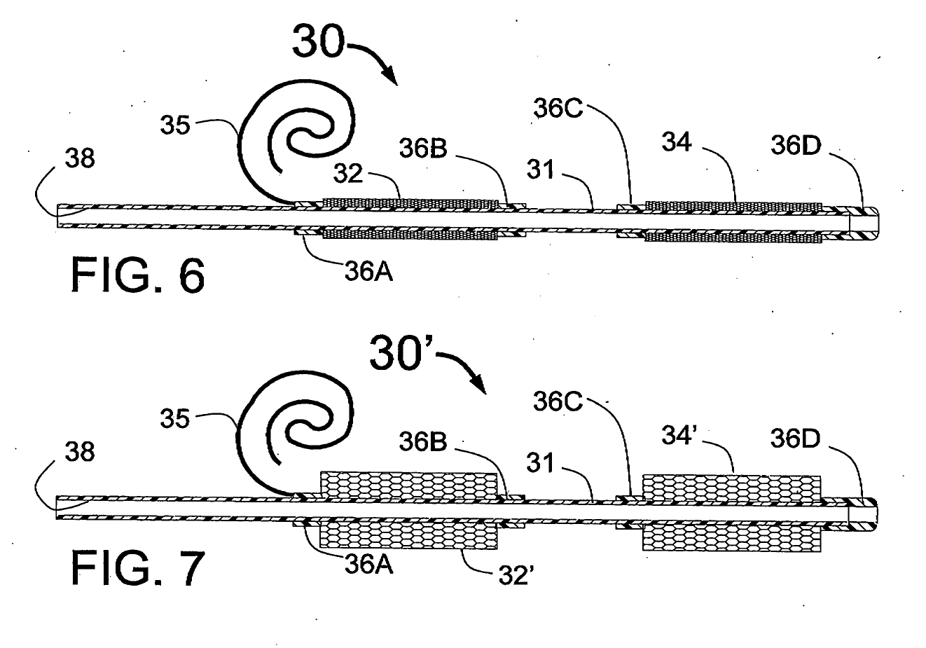

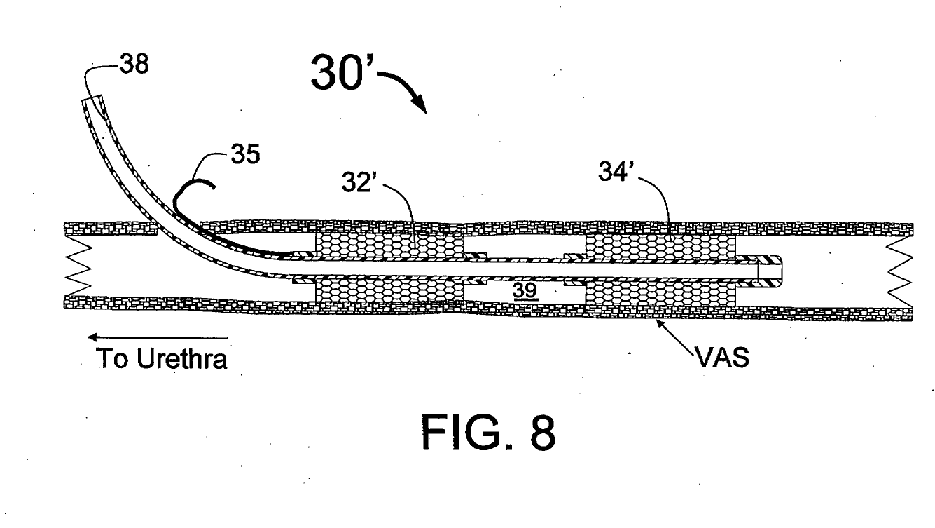

FIG. 7 is a transverse cross section of the present invention open end IVD 30 illustrating its expanded shape 30′ after deployment. (The actual post-deployment configuration within the vas and are detailed below in the discussion of FIG. 8.) The foam cylinders 32 and 34 of FIG. 6 expand to become the expanded foam cylinders 32′ and 34′ of FIG. 7.

FIG. 8 is a transverse cross section showing the open end IVD 30 after deployment in the vas and scrotum. The expandable foam cylinders 32 and 34 mounted over the flexible tube 31 are designed to adapt to the individual vas, expanding to fill the particular diameter. Thus the cylinders 32 and 34 form blockages to sperm in two places with a dead space 39 in between, but sperm is free to flow from the testes into the scrotum. The tube 31 with lumen 38 extends through the wall of the vas forming a duct to relieve fluid pressure distal to the IVD 30 and allow sperm to flow through the lumen 38 into the scrotum. The cord 35. which extends through the VAS wall typically at the side of insertion of the IVD 30, is typically sutured to the Vas to prevent migration of the IVD 30 after deployment. The cord 35 also serves the important function when and if the IVD is to be removed to reverse sterilization. To remove the IVD 30 cord 35 and/or the tube 31 are pulled proximally to remove the IVD 30 from the VAS.

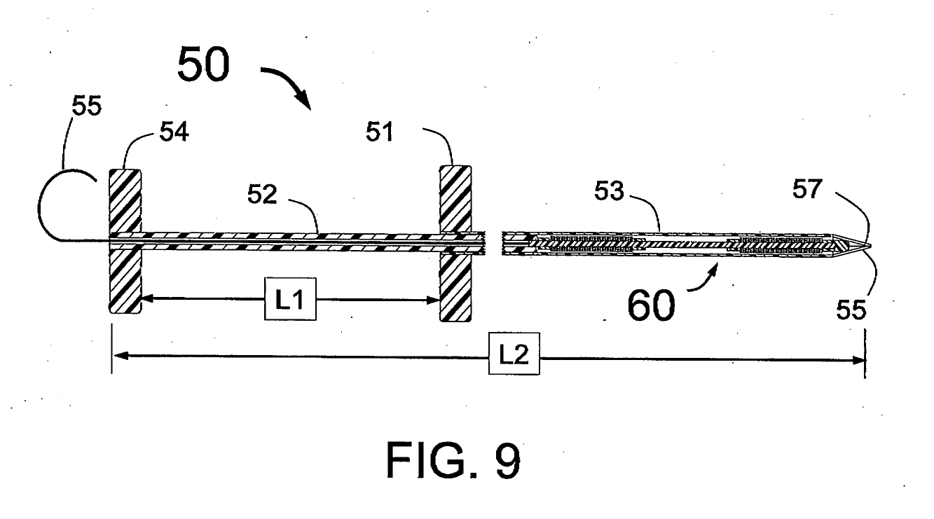

It is clear that the IVD 10 of FIGS. 2 through 5 has applications to creation of reversible blockages of other ducts of the human body. For example, when used in a woman's fallopian tubes, the Intra-Vas Device (IVD) 10 becomes an Intra-Fallopian Tube Device (IFD) 60. The most significant modification required for use in the fallopian tubes is an increase in the length of the IFD delivery system 50 shown in FIG. 9 as compared with the IVD delivery system 20 shown in FIG. 4. FIG. 9 is a transverse cross section of the pre-deployment configuration of the present invention IFD delivery system 50 having IFD 60 housed within a delivery sheath 53 with handle 51 and slit tapered end 55 with slit 57 shown. A pusher tube 52 with handle 54 provides the means of delivery of the IFD 10 into a fallopian tube. Specifically, once access to a fallopian tube is accessible, the tapered slit end 55 of the sheath 53 is inserted into the fallopian tube. There are two ways in which the IFD 60 can then be delivered. Either of these methods can be performed with vaginal delivery or with surgical access via a cut down or laparoscope.

The first method requires that the sheath 53 be advanced into the fallopian tube until the handle 51 is just proximal to the opening into the fallopian tube. At this time, the handle 51 is pulled proximally toward the handle 54 of the pusher tube 52. This will retract the sheath 53 leaving the IFD 10 inside the fallopian tube. The pusher tube 52 and sheath 53 can then be pulled over the cord 15 and removed. The cord 55 can then be left to provide a means to remove the IFD 60 to reverse sterilization.

The second method requires that the sheath 53 be advanced into the Fallopian tube until the slit tapered distal end 55 is distal to the opening into the Fallopian tube. At this time, the handle 53 is pushed distally toward the handle 51 of the sheath 53. This will push the IFD 60 distally out of the sheath 53 inside the fallopian tube. The pusher tube 52 and sheath 53 can then be pulled over the cord 55 and removed. The cord 55 can then be left to provide a means to remove the IFD 60 to reverse sterilization.

The two handles 51 and 54 are separated by the distance L1 which is typically greater than the length of the IFD 10 so that the length of the pusher tube 52 is sufficient to push the IFD 10 completely out of the sheath 53 into the fallopian tube. The entire length L2 of the IFD delivery system 50 is greater than the IVD delivery system 20 of FIG. 4 as a greater length is needed for insertion through the vagina and uterus or through a laparoscope than IVD delivery, which occurs with the vas removed completely from the scrotum. A typical length L2 would be between 20 cm and one meter.

The handles 51 and 54 and the sheath 53 are typically made from one or more biocompatible materials such as those used in angioplasty catheters. These include polymers such as urethane, nylon, polyimid and TEFLON. The pusher tube 52 can be made from plastic or a metal such as aluminum or stainless steel.

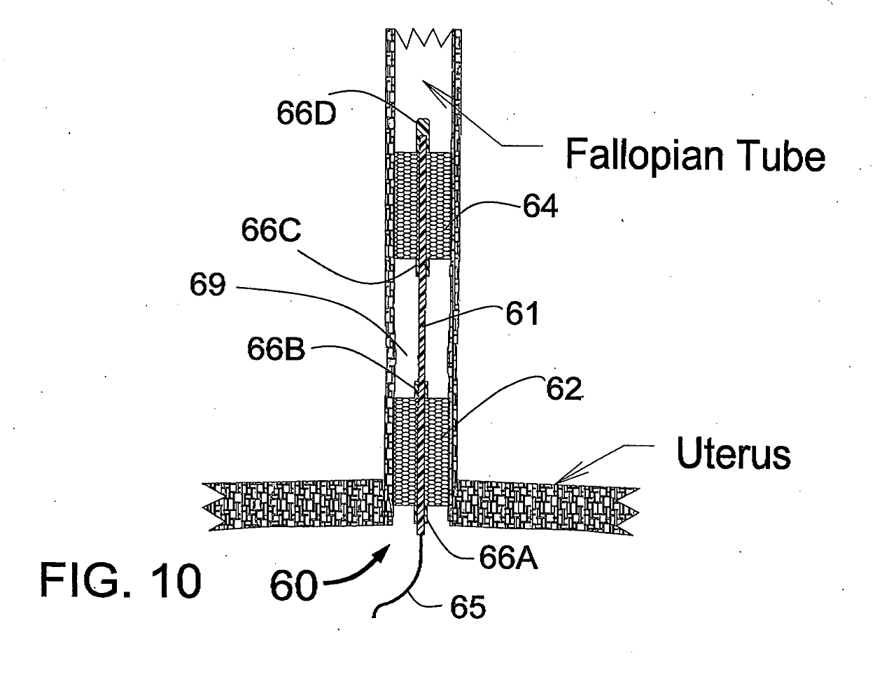

FIG. 10 is a transverse cross section of the present invention IFD 60 in its post-deployment shape placed into a fallopian tube. The IFD 60 is flexible molded plastic rod 61 with a cord 65 attached to the tail end (i.e., the uterine end, which is the end that enters the fallopian tube last). Attached to the rod 61 are two central raised portions 66B and 66C. The IFD 60 includes an expandable foam cylinder 62 inserted over the rod 61 and held in place between the stop 66A and the central raised portion 66B. The IFD 60 includes an expandable foam cylinder 64 inserted over the rod 61 and held in place between the cap 66D and the central raised portion 66C. The dead space 69 between the expanded cylinders 62 and 64 is intended to increase the effectiveness of the IFD 60 in blocking passage of eggs from ovary through the fallopian tube into the uterus and also preventing sperm from getting from the uterus into the fallopian tube, which could result in ectopic pregnancy.

The rod 61, raised portions 66B and 66C and the distal cap 66A and proximal stop 66D are typically made from one or more biocompatible materials such as those used in angioplasty catheters. These include polymers such as urethane, nylon, polyimid and TEFLON. The cord 65 would typically be made from a biostable material such as those used in permanent sutures, e.g. nylon.

The expandable sections 62 and 64 may be any biocompatible expandable material, such as a foam polymer; however the preferred embodiment of the present invention would utilize a gradually expanding hydrophilic material. The gradually expanding hydrophilic material can be any biologically compatible material such as hydrogels which are capable of expanding slowly when water is absorbed therewithin. Among the hydrogels, which are employable in the context of this invention are those utilized heretofore in cervical dilators, or in cervical devices such as described in U.S. Pat. No. 3,867,329.

Among the hydrophilic materials useful in the practice of this invention are polyvinylpyrrolidone, polyethylene glycol, karaya gum, carboxy methyl cellulose, hyaluronic acid, dextran, polyacrylic acid, polyvinyl alcohol and other organic polymers containing carboxylic acid groups or their salts. Cross-linked hydrophilic polymers or hydrogels are particularly desirable. These should be insoluble yet still capable of expanding up to 1000% due to the absorption of water. Cross-linking may be by chemical means or by physical means. The hydrogels disclosed in U.S. Pat. Nos. 3,867,329 and 4,480,642 can also be useful in this invention. Reference can also be made to G.B. 2,139,989A for suitable cross-linked polymeric compositions. Known slowly expanding dilators such as laminaria digitata or japonica can also be utilized.

Although the designs for the IVDs 10 and 30 of FIGS. 2 through 8 and the IFD 10 of FIGS. 9 and 10 show the use of two expandable foam cylinders to form the duct blockage with a dead space in between, it is envisioned that more than two expandable foam cylinders could be used. In addition, the same effect can be accomplished using two IVD or IFD devices, each with a single expandable foam cylinder. Finally it is envisioned that one longer expandable foam cylinder might be as effective as two shorter ones, particularly for application of the invention as an IFD.

It is also envisioned that the expandable cylinders 12, 14, 32, 34, 62 and 64 of the IVDs and IFDs of FIGS. 2-10 may be coated with a substance to prevent the cylinder from becoming stuck to the wall of the duct (vas or fallopian tube) into which the device is placed. The coating may be an inert substance such as silicone rubber. The coating could also be a polymer that elutes a bioactive compound such as sirolimus to prevent hyperplasia by the cells in the duct wall. It is also envisioned that instead of a coating, an anti-proliferative or anti-inflammatory compound could be loaded into the expandable cylinder and elute from the cylinders over weeks, months or years. It is also envisioned that an anti-bacterial agent can be used in conjunction with either a coating or a compound loaded into the expandable cylinder.

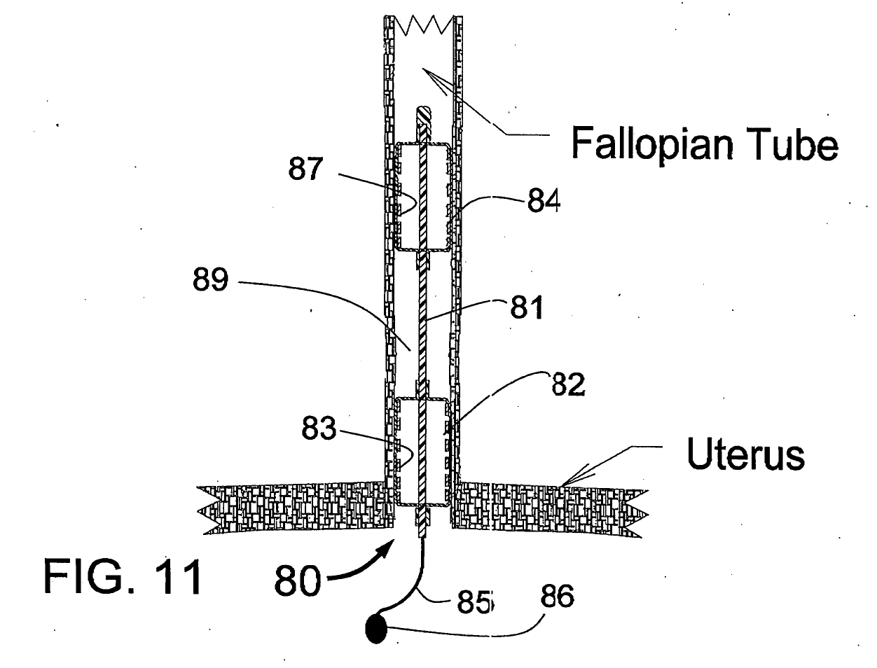

FIG. 11 shows an alternate embodiment of the present invention IFD 80. In this embodiment, the expandable foam cylinders 62 and 64 of the IFD 60 of FIG. 10 are replaced with expandable elastic cylinders 82 and 84 that are pushed outward against the wall of the fallopian tube by the self-expanding stents 83 and 87 respectively. The expandable elastic cylinders 62 and 64 are mounted onto the rod 81 and, when expanded, create a dead space 89 between the expanded elastic cylinders 62 and 64, which serves the same purpose as the dead space 69 of FIG. 10. The expandable elastic cylinders 62 and 64 can be made from any biocompatible elastic polymer, which is elastic enough to be expanded outward by the self-expanding stents 83 and 87. For example the expandable elastic cylinders 82 and 84 could be made of medical grade silicone rubber. The self-expanding stent would typically be made from nitinol having a transition temperature slightly below body temperature. The pull cord 85, having an optional pull tab 86 attached to its tail end, can be used to remove the IFD 80 from the fallopian tube to reverse the sterilization of the patient. The entire IFD 80 after it is built would be squeezed down and placed inside the IFD delivery system 50 of FIG. 9. The IFD 80 of FIG. 11 would be delivered into the fallopian tube using the same technique as the IFD 60 of FIGS. 9 and 10. It is also envisioned that the expandable elastic cylinders 82 and 84 could be hydrophylically coated to assist in removal, or the cylinders 82 and 84 might be coated with a compound that elutes one or more drugs. Such drugs include anti-inflammatory agents, anti-bacterial agents and anti-proliferative agents such as sirolimus, paxital or everolimus.

Various other modifications, adaptations, and alternative designs are of course possible in light of the above teachings. Therefore, it should be understood at this time that, within the scope of the appended claims, the invention can be practiced otherwise than as specifically described herein.

What is claimed is:

1. A implantable medical device designed to be inserted into a duct of the human body, the device having an insertion end and a tail end, the device also having at least two expandable portions that are separated longitudinally one from the other, each of the expandable portions being designed to block the flow of fluid through the duct.

2. The implantable medical device of claim 1, further including means to remove the device from within the duct so as to restore fluid flow.

3. The device of claim 2, where the means to remove the device comprises a structure attached to the tail end of the device.

4. The device of claim 3, where the structure includes a pull cord.

5. The device of claim 4, where the structure includes a pull tab attached to the pull cord.

6. The device of claim 1, where the two expandable portions are separated by more than 1 millimeter.

7. The device of claim 1, where the two expandable portions are separated by less than 2 centimeters.

8. The device of claim 1, where the two expandable portions are made from an expandable foam material.

9. The device of claim 8, where the expandable foam material is polyvinyl alcohol.

10. The device of claim 8, where the expandable foam material is includes at least one substance from the list comprising:
a. Polyvinylpyrrolidone;
b. polyethylene glycol;
c. carboxy methyl cellulose;
d. karaya gum;
e. hyaluronic acid;
f. dextran;
g. polyacrylic acid;
h. organic polymers containing carboxylic acid groups or their salts;
i. cross-linked hydrophilic polymers; and
j. hydrogels.

11. The device of claim 1 where the expandable portions are coated with at least one substance selected from the list comprising:
a. an anti-bacterial compound;
b. an anti-inflammatory compound;
c. an anti-proliferative compound;
d. sirolimus or one of its analog cytostatic compounds;
e. paxitaxel or one of its analog compounds; and
f. a hydrophyllic compound.

12. The device of claim 1, where the expandable portions have cylindrical shape.

13. The device of claim 1, where the expandable portions have a maximum expanded diameter of less than 5 millimeters.

14. The device of claim 1, further including a delivery system adapted to deliver the device into the lumen of the vas of a human patient.

15. The device of claim 1, further including a an intra-vaginal delivery system adapted to deliver the device into the fallopian tube of a human patient.

16. The device of claim 1, further including a shunt to divert the fluid that would otherwise flow through the duct to an alternative location.

17. The device of claim 1, further including means to secure the device to fix its position within the duct.

18. The device of claim 17, where the means to secure the device is a suture.

19. The device of claim 1, where the expandable portions include a self-expanding stent.

20. The device of claim 1, further including a delivery sheath that holds the device in an unexpanded state and from which the device is delivered into the duct where it expands to an expanded state.
